(19) United States
(12) Patent Application Publication
(10) Pub. No.: US 2021/0395662 A1
(43) Pub. Date: Dec. 23, 2021

(54) Cell Culture Device

(71) Applicant: Institut Mines-Télécom, Palaiseau (FR)

(72) Inventors: Mikhael Hadida, Saint Etienne (FR); David Marchat, Saint-Etienne (FR)

(73) Assignee: Institut Mines-Télécom, Palaiseau (FR)

(21) Appl. No.: 17/292,245

(22) PCT Filed: Nov. 8, 2019
(86) PCT No.: PCT/FR2019/052669

§ 371 Date: May 9, 2021

(30) Foreign Application Priority Data
Nov. 9, 2018 (FR) 1860401

(51) Int. Cl. C12M 1/12; C12M 1/00; C12M 3/06; C12M 3/00; C12M 1/34

(52) U.S. Cl. CPC C12M 25/14; C12M 23/22; C12M 41/36; C12M 23/48; C12M 23/16

(57) ABSTRACT

Cell culture and/or seeding device (10) comprising:

at least one culture chamber having at least one transparent optical window (11),

at least one fluid inlet (12) and at least one fluid outlet (13) communicating with the culture chamber,

at least one three-dimensional culture scaffold (20) arranged in the culture chamber, having surfaces (21, 22) spaced out along an axis (X) of the culture scaffold, the culture scaffold being arranged in the culture chamber in such a way as to be traversed from one surface to the other by the flow circulating between the fluid inlet and the fluid outlet, and in such a way that one of the surfaces thereof, preferably the fluid inlet face, is situated at least partially facing the optical window (11).

The present invention relates to cell culture and/or seeding methods and devices.

Perfused bioreactors are culture systems that comprise a culture scaffold swept by a culture medium.

An example of such a bioreactor is that sold by the Cellec Biotek Company under the reference U-CUP. It comprises a three-dimensional culture scaffold placed in a chamber traversed by a culture medium imbued with an oscillating movement.

Such a device does not enable observation of cell behaviour by optical microscope without extracting the culture scaffold from the chamber. It thus does not make it possible to monitor in real time cell behaviour within the culture scaffold. Optically transparent is taken to mean a material faithfully transmitting an optical ray. Preferentially, it is taken to mean a material faithfully transmitting an optical ray in the visible spectrum.

Patent application US 2016/0040108 discloses a bioreactor comprising a culture scaffold trapped between two parts ensuring the distribution of flows of incoming and outgoing fluid, maintained assembled together by a cylinder. This device is compatible with a non-optic imaging technique such as CT (Computed Tomography) or MRI (Magnetic Resonance Imaging).

Patent application US 2013/0344531 discloses a device enabling optical examination of a cell culture in vitro. The device comprises a chamber having a transparent window behind which is arranged a culture scaffold. Said scaffold is in the form of a disk of small thickness arranged between two parts of annular shape, of which one is traversed by diametrically opposite radial orifices enabling the circulation of a nutrient medium therebetween. The culture scaffold may thus be swept by the nutrient medium. Such a device is not provided to receive a three-dimensional culture scaffold, thicker, requiring circulation of the nutrient medium in its thickness.

Patent application WO2015/134550 discloses a culture device comprising micro-channels to ensure the circulation of the nutrient medium through multiple perfusion chambers. Such a device is not provided for observation of the culture scaffold by optical microscopy, without having to carry out the dismantling thereof.

Patent application US 2011/0229970 describes a bioreactor making it possible to direct the flow of liquid in an adjustable manner with respect to the culture scaffold.

Patent application WO2013/103306 describes a bioreactor comprising a three-dimensional culture scaffold. However, the design of this device and the mode of insertion of the culture scaffold do not make it possible to control the circulation of the nutrient medium within the culture scaffold. In other words, this device does not enable a control of the fluidic environment within the culture scaffold.

There exists a need to benefit from a cell culture device which enables observation of cell development by optical means, while offering conditions of circulation of the nutrient medium suitable for the culture of cells sensitive to their fluidic environment, such as cells grown by tissue engineering. For such cells, it is important to place the cells in physiologically relevant configurations.

There also exists an interest to have available a cell seeding device which makes it possible to seed a culture scaffold, notably in a homogeneous manner, and to verify optically the correct seeding thereof.

The invention aims to respond to all or part of these needs and it manages to do so thanks to a cell culture and/or seeding device comprising:
at least one culture chamber having at least one transparent optical window,
at least one fluid inlet and at least one fluid outlet communicating with the culture chamber,
at least one three-dimensional culture scaffold arranged in the culture chamber, having surfaces spaced out along an axis of the culture scaffold, the culture scaffold being arranged in the culture chamber in such a way as to be traversed from one surface to the other by the flow circulating between the fluid inlet and the fluid outlet, and in such a way that one of the surfaces thereof, preferably the fluid inlet surface, is situated at least partially facing the optical window.

Preferably, the device comprises at least one network of micro-channels arranged in series with the culture scaffold, preferably situated upstream of the culture scaffold, this network being connected for example to the fluid inlet, and comprising a plurality of outlets for supplying the culture scaffold.

“Culture scaffold” designates any material enabling cells to be seeded and cell development. The culture scaffold may if necessary be transplanted or implanted, in which case it constitutes an implant.

The invention makes it possible to carry out a cell culture in a fluidic environment enabling the control of shear forces and circulation speeds in the culture scaffold.

In particular, the invention makes it possible, if so wished, to lay out the network of micro-channels in such a way as to have a relatively homogeneous distribution of the flow rate at the outlet of this network. This makes it possible to have a relatively homogeneous flow rate at the outlet of the culture scaffold. Moreover, this also makes it possible to have a homogeneous flow rate through the culture scaffold.

The invention enables optical examination, for example by confocal microscope, of the culture scaffold without interruption of the perfusion.

The invention may be used, among other applications, within the scope of understanding the biology of bones, notably the study of the mechanical-transduction responses of bone cells and that of the impact of biochemical factors on bone cells, with the aim notably of optimising the production of living bone grafts.

The invention also offers the possibility of having available bone models as a standard platform for screening molecules, notably osteo-active or anti-cancerous molecules, as replacement for animal models.

The invention also makes it possible to study the impact of numerous culture parameters on the development of cells and tissue.

The invention enables homogeneous seeding of the culture scaffold, great facility of implementation and great repeatability of tests.

Preferably, the device comprises a collector leading into the outlet face of the culture scaffold, connected to the fluid outlet. This collector makes it possible, by playing on its geometry, to exert an additional control on flows through the culture scaffold.

Preferably, the optical window is of low thickness, for example less than or equal to 1 mm. This enables good visualisation of the culture scaffold from the outside of the device.

In an exemplary embodiment of the invention, the optical window is defined by a plate directly mounted on a body having a housing to receive the culture scaffold. The plate used is preferably made of a material of optical quality, such as optical quality PMMA. Such a plate has for example a high degree of transparency, as well as a low thickness, which is useful for not unduly moving the culture scaffold away from the optical instrument.

In an alternative, the optical window is produced monolithically with at least a part of the body that defines the housing for receiving the culture scaffold. This optical window may be produced at least partially monolithically with the aforesaid network of micro-channels.

Preferably, the culture scaffold is removably received in the culture chamber. This makes it possible to mount a large variety of culture scaffolds in the device. In other words, this makes it possible to adjust a large variety of culture scaffolds in the chamber.

The device may comprise a seal arranged around the culture scaffold. This obliges the fluid to traverse entirely the culture scaffold and avoids any risk of peripheral flow. This seal may have various shapes. It may be advantageous to use a seal having an inclined peripheral surface so that an axial tightening of the seal is accompanied by a radial tightening against the culture scaffold.

As mentioned above, the network of micro-channels may be laid out to generate relatively homogeneous flow at the inlet of the culture scaffold. The network of micro-channels may thus be configured to deliver a same flow rate to some 10%, on each of its outlets. Within the culture scaffold, and at the outlet thereof, the flow rate is for example controlled with a precision equal to or better than 2%. The culture scaffold may thus have channels, and the flow rate at the outlet of these channels may be homogeneous to better than 2%, that is to say that if dmin is the minimum flow rate observed at the outlet of a channel, and dmax the maximum flow rate, (dmax-dmin)/dmin is less than or equal to 0.02.

The outlets of the network of micro-channels may have axial symmetry or rotational symmetry, notably second order symmetry, better fourth order, even better eighth order at least. The network of micro-channels may comprise at least two stages of multiple ramifications.

Preferably, the network of micro-channels extends along a plane perpendicular to the axis of the culture scaffold. It may comprise channels that extend around the optical window. Said window may be centred relative to the network of micro-channels.

The device may comprise a block having a housing defining at least partially the culture chamber, having a bearing face against which a bottom plate is directly mounted, the network of micro-channels being formed between said face and the bottom plate, the micro-channels preferably being formed as hollows on said bearing face. The optical window may be formed by this bottom plate. The block may form the aforesaid body. The invention is not however limited to this particular way of producing the optical window.

Preferably, the collector has a conical surface turned towards the outlet face of the culture scaffold. The shape of the collector can influence the flow speeds within the culture scaffold. It is thus possible to play on the shape of the collector to improve the homogeneity of fluid flow within the culture scaffold. The collector may further have a cylindrical shape or other, for example staged.

The culture scaffold may have any outer shape and for example an outer shape having axial symmetry with respect to its axis. The culture scaffold may have a generally cylinder of revolution outer shape. In an exemplary embodiment, the culture scaffold comprises a plurality of parallel channels, extending between the inlet and outlet surfaces of the culture scaffold, these channels preferably having a circular section, the channels preferably being parallel to the axis of the culture scaffold. The inlet and outlet surfaces may be flat faces.

The culture scaffold may also have a tri-periodic structure, of gyroid type.

The shape of the inlet and outlet surfaces may be diverse, being for example flat and perpendicular to the axis of the culture scaffold or other.

The axial dimension of the scaffold may be comprised between 0.2 mm and 20 mm, better 1 mm and 10 mm.

The diameter of the culture scaffold, that is to say the largest transversal dimension thereof, may be comprised between 3 mm and 20 mm, or better 5 mm and 12 mm.

The volume of the culture scaffold may be comprised between 3.5 mm³ and 6300 mm³, better between 20 mm³ and 1130 mm³

The fluid inlet and the fluid outlet may be arranged on a same side of the culture chamber, preferably the upper side thereof. In this case, the optical window is advantageously situated on the lower side of the device. This enables the tubing connected to the fluid inlet and fluid outlet not to interfere mechanically with the optical instrument placed facing the optical window.

The device may comprise a body defining a housing in which is arranged the culture scaffold, and an insert to close at least partially said housing, this insert preferably being screwed into the body. The insert may comprise an end lip engaged on the seal, and extending preferably over a part only of the height of the seal. The insert may define the aforesaid collector. The insert may have a central cut-out to receive an end fitting for connecting a departure or outlet tubing, this end fitting preferably being screwed into the insert.

In a preferred exemplary embodiment of the invention, the lip of the insert has a conical radially inner surface, and the seal has a conical radially outer surface, substantially of same slope, such that the axial displacement of the insert during its tightening is accompanied by wedge effect with tightening of the seal on the culture scaffold. The seal has for example a radially outer surface inclined with respect to its axis of symmetry, preferably a rectangular trapezoid shaped section.

The subject matter of the invention is further a system comprising a culture and/or seeding device according to the invention, such as defined above, and a fluidic circuit for circulating a culture medium in the culture chamber, this culture medium entering the culture chamber via said fluid inlet and leaving it via said fluid outlet, said fluidic circuit preferably being laid out to establish circulation or recirculation of the culture medium through the culture chamber.

The circulation of fluid may take place always in the same direction, for example firstly through the network of micro-channels then the culture scaffold. In an alternative, the circulation takes place in the opposite direction, namely firstly through the culture scaffold then through the network of micro-channels. In particular, the circulation may be carried out in an alternating manner in one direction then in the other during a seeding phase of the culture scaffold, and next in a unidirectional manner during a cell development phase.

The system may comprise at least one sensor in the outlet of the culture chamber, this sensor preferably being capable of measuring dissolved dioxygen, pH, glucose, lactate, or other metabolites and specific signals generated by the activity of cells and more particularly bone cells, such as osteoblastic markers (e.g. alkaline phosphatase and osteocalcin) and osteocyte markers (e.g. sclerostin), or adipocytes with for example adiponectin markers and FABP4.

The system may comprise a microscope, preferably confocal, arranged so as to observe the culture scaffold, the observation taking place through said optical window.

The system may comprise at least two culture devices according to the invention, connected in series.

The subject matter of the invention is further a cell culture and/or seeding method, in which cells are seeded and potentially grown, for example stem cells, or bone cells, notably osteoblasts, osteoclasts or osteocytes, on a culture scaffold of a device according to the invention, or a system according to the invention.

The invention may be implemented to differently differentiate stem cells as a function of culture conditions.

When the system comprises two devices in series, said devices may be seeded with cells of different types or not.

The culture scaffold may also be seeded with at least two types of cells to produce a co-culture.

Preferably, seeding, culture, sampling and on-line analyses are carried out in an automated manner.

The subject matter of the invention is further, according to another of its aspects, independently or in combination with the preceding, a cell culture and/or seeding device comprising:
at least one culture chamber,
at least one fluid inlet and at least one fluid outlet communicating with the culture chamber,
a three-dimensional culture scaffold arranged in the culture chamber, having surfaces spaced out along an axis of the culture scaffold, the circulation of the fluid between the inlet and the fluid outlet taking place between said surfaces,
a network of micro-channels situated upstream of the culture scaffold, this network being connected to the fluid inlet and comprising a plurality of outlets for supplying the culture scaffold, this network of micro-channels extending along a plane perpendicular to the axis of the culture scaffold.

Such a relative arrangement of the culture scaffold and the network of micro-channels makes it possible to minimise the axial bulk of the device, notably facing the inlet face of the culture scaffold. This also makes it possible to shift the supply of the network of micro-channels onto the side of the device.

The network of micro-channels may have all or part of the aforesaid characteristics, and notably comprise several ramification stages, multiple outlets having for example axial symmetry or second order or more rotational symmetry around the axis of the culture scaffold. The network comprises, in an exemplary embodiment, channels formed as hollows in a block and closed by a bottom plate. In an alternative, the channels are formed otherwise, for example by an additive manufacturing technique.

The invention will be able to be better understood on reading the description that follows of non-limiting exemplary embodiments thereof, and by examining the appended drawing, in which:

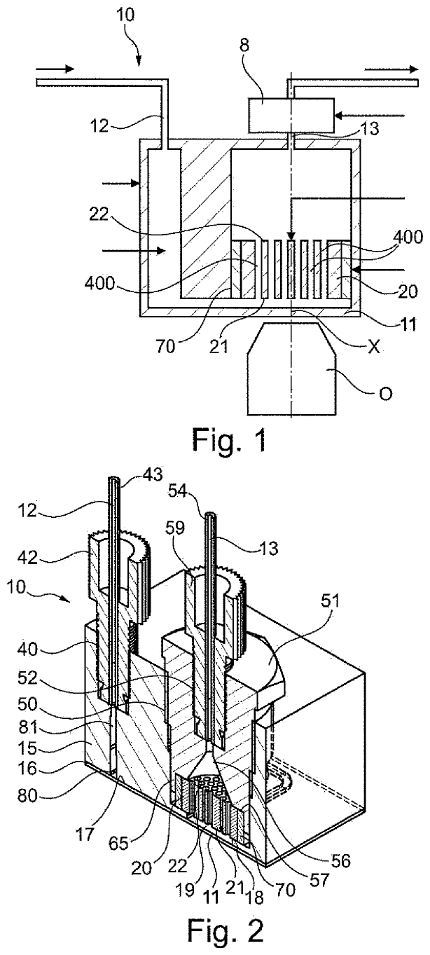

FIG. 1 represents in a schematic manner in section an exemplary device according to the invention, as well as an associated optical instrument,

FIG. 2 is a perspective view with axial section of a more detailed exemplary embodiment of the device,

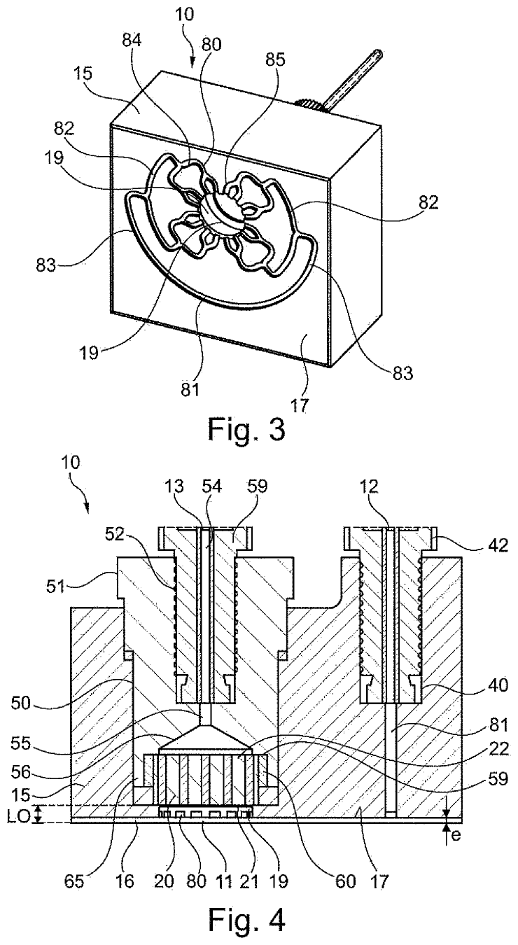

FIG. 3 represents in isolation, in bottom view, the body of the device of FIG. 2,

FIG. 4 illustrates, in axial section, the assembly of certain constituent parts of the device of FIGS. 2 and 3,

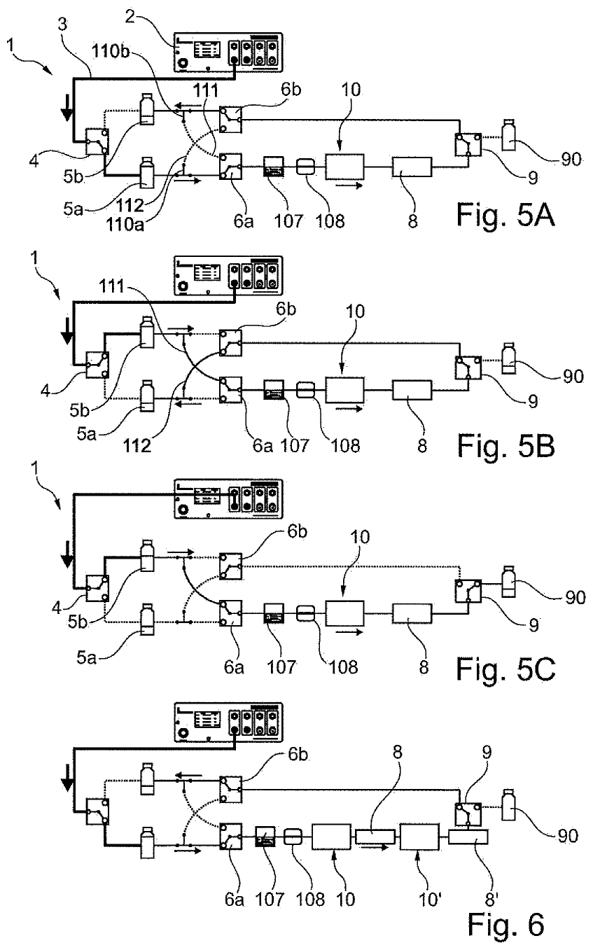

FIGS. 5A to 5C represent an exemplary system using a device according to the invention,

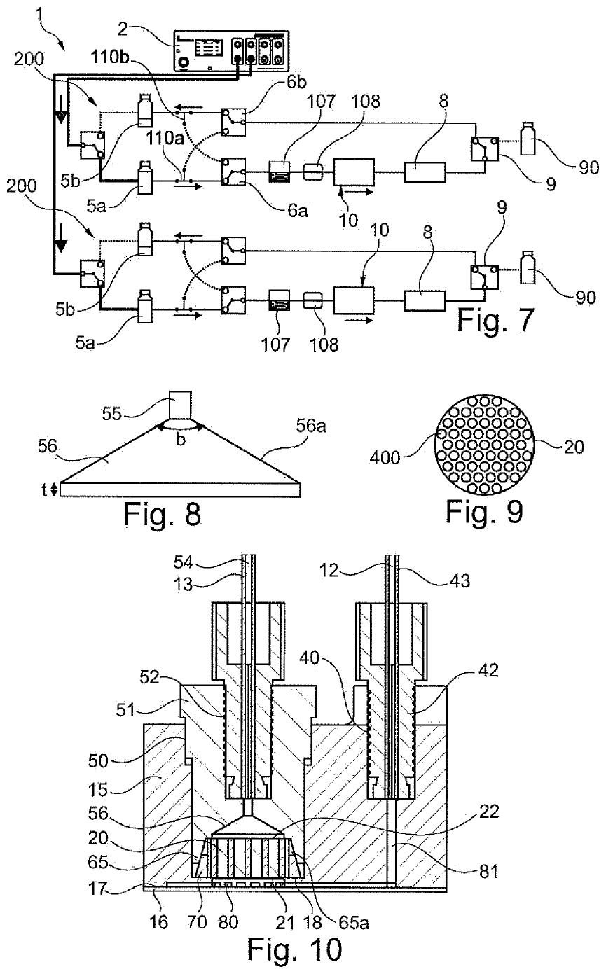

FIGS. 6 and 7 are views analogous to FIGS. 5A to 5C of alternatives of systems according to the invention,

FIG. 8 illustrates an exemplary collector geometry,

FIG. 9 represents an exemplary distribution of channels within the culture scaffold, and

FIG. 10 is a view analogous to FIG. 4 of an alternative embodiment of the invention.

A cell culture and/or seeding device 10 according to the invention (also called bioreactor) comprises, as illustrated in a simplified and schematic manner in FIG. 1, a cell culture scaffold 20, arranged in a culture chamber in such a way as to be able to be traversed by a flow of a nutrient medium.

The culture scaffold is three-dimensional, has an inlet surface 21 and an outlet surface 22 both spaced out along an axis X, and in the example illustrated each of flat shape and perpendicular to the axis X. Fluidic circulation may be established between the inlet 21 and outlet 22 surfaces, along the axis X, due to the porosity of the scaffold.

The culture scaffold 20 has for example parallel channels 400 of circular section, which makes it possible to expose the whole of the culture surface to a substantially constant shear value, and thus to identify more easily cell response to mechanical stimulation thresholds. The culture scaffold may further have a gyroid type tri-periodic structure.

In FIG. 9 is represented an exemplary distribution of the channels 400 which has a good ratio between seeding surface and homogeneity of shear forces.

In accordance with the invention, the device comprises an optical window 11, arranged at least partially facing the fluid inlet surface 21 of the culture scaffold.

The device comprises at least one fluid inlet 12 and one fluid outlet 13, between which the circulation of the fluid takes place.

A sealing member 70 may be arranged in the culture chamber around the culture scaffold 20, as illustrated.

In the example considered, the inlet 12 and the outlet 13 are situated opposite the optical window 11, which facilitates the putting in place of the device 10 with the optical window 11 facing an instrument O, such as a confocal microscope, as illustrated.

The inlet 12 may be shifted laterally from the optical axis of the instrument O, which may be merged with or parallel to the axis X of the culture scaffold 20.

The outlet 13 of the device may be connected to one or more sensors 30, to analyse the medium having perfused through the culture scaffold 20.

In FIGS. 2 to 4 is represented in a more detailed manner an exemplary embodiment of the device 10 according to the invention.

The device 10 comprises a body 15 produced for example by machining of a thermoplastic material compatible with biological applications, for example PMMA (polymethylmethacrylate), PDMS (polydimethylsiloxane) or PS (polystyrene), closed inferiorly by a bottom plate 16 made of a transparent plastic material and of which the region positioned facing the culture scaffold defines the optical window 11. The bottom plate 16 is for example made of optical quality PMMA, and the thickness e thereof is preferably relatively low, for example less than or equal to 1 mm or 0.5 mm.

The device 10 may be produced otherwise, while preferably ensuring that the optical window remains of relatively low thickness, for example less than or equal to 1 mm or better less than or equal to 0.5 mm.

The body 15 comprises a first housing 40 laid out to receive an end fitting 42 for connecting a tubing 43 for feeding fluid to the inlet 12 of the device.

The body 15 comprises a second housing 50 for receiving an insert 51, which is made of a material compatible with the cell culture, for example the same material as the body 15, and screwed into the housing 50. This housing inferiorly leads into the lower face 17 of the body 15 via an opening 19, for example of circular contour as illustrated.

The insert 51 has a housing 52 which superiorly leads into, and in which is fixed, an end fitting 59 for connecting a tubing 54 to the fluid outlet 13 of the device.

The housing 52 communicates inferiorly by a channel 55 with a collector 56 which opens out onto the culture scaffold 20.

The latter is received in a housing 60 of the insert 51, open downwards, delimited at its periphery by an annular lip 65 of same axis as the culture scaffold 20.

A ring seal 70, visible in FIG. 2, is interposed radially between the lip 65 and the culture scaffold 20. This seal 70 is for example made to measure out of medical grade silicone, and has any shape suited to obtaining the desired sealing.

In FIG. 2 the seal has a cylindrical shape. Preferably, the seal 70 is produced with a rectangular trapezoid shaped section, as illustrated in FIG. 10, and the lip 65 of the insert has a conical radially inner surface 65a converging towards the base of the lip. This surface bears against a conical surface of the seal 70, of same slope, such that an axial displacement of the lip 65 during screwing of the insert 51 is accompanied by radial tightening of the seal 70 against the culture scaffold, by wedge effect. The sealing may further be realised otherwise, without going beyond the scope of the invention.

A network 80 of micro-channels is produced as hollows on the lower face 17 of the body 15.

This network 80 communicates with a feed channel 81 formed in the extension of the housing 40.

The network 80 comprises several successive ramification stages, namely a first stage comprising two arc of circle branches 82 connecting to the feed channel 81 by channels 83.

Each canal 82 communicates at one of the ends thereof with a second stage comprising two branches 84.

Each branch 84 communicates at one end with a third stage comprising two branches 85, which lead into the opening 19, at the periphery thereof.

Each pair of branches 85 of the third ramification stage is the image of another pair by a rotation of k*360°/8 around the axis of the housing 50, where k is an integer comprised between 1 and 7.

The network of micro-channels that supplies the culture scaffold may comprise, in an alternative not illustrated, a larger number of outlets.

The bottom plate 16 may be bonded onto the face 17 while taking care not to close off the network 80 or opacify the optical window 11. The bottom plate may further be fixed otherwise. Thus, according to other embodiments, the bottom plate 16 and the face 17 could be a single and same one-piece element, optically transparent (such as PMMA).

Due to the thinness of the optical window and the low thickness of the wall 18 defining the bottom of the housing 50 around the opening 19, the lower face 21 of the culture scaffold is situated at a relatively small distance w from the lower face of the device, as may be seen in FIG. 4, which facilitates observation of the culture scaffold 20 by microscope through the optical window 11.

The geometry of the collector 56 influences the distribution of shear forces within the channels 400 of the culture scaffold 20. The collector 56 preferably has, as illustrated in FIG. 8, a converging conical surface 56a on moving away from the outlet face 22 of the culture scaffold 20. The angle b at the top is preferably greater than 90°,being notably comprised between 90° and 180° and more particularly between 90° and 150°, being for example 120°. The presence of a conical surface makes it possible to homogenise flow within the culture scaffold 20 with respect to the outlets 85 of the network of micro-channels.

The conical surface 56a may extend in the direction of the culture scaffold by a cylindrical surface of revolution 56b, over a distance t, the latter preferably being comprised between 0 and 2 mm (terminals excluded), for example 0.5 mm.

Conical geometry is only an exemplary embodiment of the invention. In the case of a geometry of revolution other than conical, for example a dome or cylinder shape, the dimensions of the surface in the axis X and notably the dimension t, may be different, and for example go from 0 to 20 mm, being for example 5 mm.

The device 10 according to the invention may be used within a micro-fluidic system 1 such as that illustrated in FIGS. 5A to 5C.

This system 1 may comprise an automaton 2 having a programmable pressure outlet 3. The automaton 2 is for example that sold by the Elveflow Company.

A first three way valve 4 makes it possible to send the pressure of the gas delivered by the automaton either to a first reservoir 5a, or to a second reservoir 5b.

Two sets of three way valves 6a and 6b complete the system 1. One of these valve ways is connected to a reservoir and the other, through a T connector 110a or 110b, to the other of the reservoirs.

More specifically, one of the inlets of the valve 6a is connected through a connector 110a to the first reservoir 5a, whereas the other inlet is connected by a duct 111 to the connector 110b. One of the outlets of the valve 6b is connected through the connector 110b to the reservoir 5b whereas the other outlet is connected by a duct 112 to the connector 110a. In FIG. 5A, the ducts 111 and 112 are represented in dotted lines, to materialise the fact that they are not traversed, in the illustrated configuration of the valves 6a and 6b, by any flow. More generally, in FIGS. 5A to 5C, are represented in solid line a duct traversed by a flow, and in dotted line an inactive duct.

The outlet of the valve 6a may be connected through a flowmeter 107 then a bubble trap 108 to the inlet of the device 10. The flowmeter 107 may supply a return signal to the automaton 2 in order to allow it to respect a set flow rate value by modulating the gas pressure, for example. The location of the flowmeter 107 is different, in an alternative.

The outlet of the device 10 is connected to one or more sensors 8 then to the inlet of a three way valve 9. This or these sensors make it possible for example to assay dissolved dioxygen, pH, glucose, lactate, or other metabolites and specific signals generated by the activity of cells and more particularly bone cells, such as osteoblastic markers (e.g. alkaline phosphatase and osteocalcin) and osteocyte markers (e.g. sclerostin), or adipocytes with for example adiponectin markers and FABP4.

One of the outlets of this valve 9 is connected to a sample collection bottle 90, and the other of the outlets is connected to the inlet of the valve 6b.

Preferably all the aforesaid valves are manageable automatically, which enables automatic operation.

With the position of the valves 4, 6a, 6b and 9 illustrated in FIG. 5A, the pressure delivered by the automaton to the reservoir 5a pushes the liquid contained therein towards the valve 6a and the device 10. The fluid coming out of the latter enters through the valves 9 and 6b the reservoir 5b, which fills up.

Next, the position of the valves 4, 6a and 6b may be reversed, as illustrated in FIG. 5B. The pressure pushes the liquid contained in the reservoir 5b towards the device 10, whereas the return of the fluid takes place in the reservoir 5a.

In perfusion regime through the culture scaffold 20, fluid circulates from bottom to top in the device 10, and observation within the volume of the culture scaffold 20 can be realised in real time by confocal microscope without interrupting the circulation of fluid nor having to extract the culture scaffold 20. It is thus possible to observe, for example, tissue growth. The seal 70 ensures perfect perfusion of the culture scaffold 20. In parallel with observation of the culture scaffold 20, the sensor or sensors 8 enable the monitoring of cell activity parameters, without human intervention. The device 10 makes it possible to ensure precise control of the fluidic parameters traversing the culture scaffold 20.

To collect a sample, the valve 9 is actuated to direct the flow coming out of the device 10 towards the bottle 90, as illustrated in FIG. 5C.

It is possible to place several devices according to the invention in series, for example at least two, such that the flow coming out of one enters the inlet of the other. This makes it possible to study the effects of secretions of cells present on the culture scaffold of the upstream device on those of the culture scaffold of the device situated downstream.

As an example, in FIG. 6 is represented a system comprising two devices 10 and 10′ according to the invention, for example structurally identical, the system being identical to that of FIGS. 5A to 5C apart from the presence of the second device 10′ connected in series to the outlet of the sensor or sensors 8 and the presence of one or more additional sensors 8′ between the outlet of the second device 10′ and the valve 9 serving for the collection of samples.

It may thus be interesting to place in series a device according to the invention with a bone culture, with a cell culture of another organ or tissue in order to study interactions between bone cells and the cells of this other organ or tissue.

The device according to the invention may thus be arranged in series with cells of:
Muscle; see the publication “Bone and muscle: Interactions beyond mechanical” [Brotto et al. 2015];
Liver; see “Bone disorders in chronic liver disease” [Collier 2007];
Kidney; see “Mineral and bone disorders in chronic kidney disease and end-stage renal disease patients: new insights into vitamin D receptor activation” [Boyer et al. 2011];
Large intestine; see “The role of the gastrointestinal tract in calcium homeostasis and bone remodeling” [Keller et al. 2013], “Understanding the Gut-Bone Signaling Axis” [McCabe et al. 2017];
Stomach; see “Stomach and Bone” [McCabe et al. 2017];
Pancreas; see “Skeleton and glucose metabolism: a bone-pancreas loop” [Faienza et al. 2015];
Parathyroid gland; see “Bone Health and Osteoporosis: A Report of the Surgeon General” [Rockville 2004];
Adrenal gland; see “Bone Health and Osteoporosis: A Report of the Surgeon General” [Rockville 2004];
Soft tissue, e.g. adipose cells and lymphoid tissue; see “Bone Health and Osteoporosis: A Report of the Surgeon General” [Rockville 2004], and “Osteocytes Regulate Primary Lymphoid Organs and Fat Metabolism” [Sato et al. 2013]

In FIG. 7 is illustrated the possibility of arranging several culture sub-systems 200 in parallel, with pooling of certain elements such as for example the automaton 2, when it is multi-path.

To carry out seeding, it is possible to control the valves so as to make the fluid circulate alternately in one direction then in the other through the culture scaffold 20.

The invention is not limited to the examples that have been described above. For example, observation of the culture scaffold 20 may be carried out by other imaging techniques, for example OCT.

Finally, with the invention, it is possible to have available a precise and reliable tool enabling a large number of parameters to be adjusted, and in particular the flow conditions (i.e.

the fluidic environment). The device is simple to use, reproducible, enables seeding of the culture scaffold, renewal of the medium and/or collection of samples in an automated manner.

The device according to the invention is also polyvalent, making it possible to receive not just synthetic culture scaffolds but also explants. The culture scaffold may have another geometry.

The fluidic system within which the device according to the invention is installed may not function with recirculation of the medium.

It is possible to integrate several types of cells within a same culture scaffold (co-culture) in order to study for example the effects of contact between cells.

1. Cell culture and/or seeding device (10) comprising:
at least one culture chamber having at least one transparent optical window (11),
at least one fluid inlet (12) and at least one fluid outlet (13) communicating with the culture chamber,
at least one three-dimensional culture scaffold (20) arranged in the culture chamber, having surfaces (21, 22) spaced out along an axis (X) of the culture scaffold, the culture scaffold being arranged in the culture chamber in such a way as to be traversed from one surface to the other by the flow circulating between the fluid inlet and the fluid outlet, and in such a way that one of the surfaces thereof, preferably the fluid inlet face, is situated at least partially facing the optical window (11),
at least one network (80) of micro-channels situated in series with the culture scaffold (20).
a seal (70) arranged around the culture scaffold (20).

2. Device according to claim 1, the network of micro-channels being situated upstream of the culture scaffold (20), this network being connected to the fluid inlet and comprising a plurality of outlets (85) for supplying the culture scaffold.

3. Device according to one of the preceding claims, comprising a collector (56) leading into an outlet surface of the culture scaffold (20), connected to the fluid outlet (13), the collector (56) preferably having a conical surface (56a) turned towards the culture scaffold.

4. Device according to any one of the preceding claims, the optical window (11) being defined by a plate (16) directly mounted on a body (15) having a housing (50) to receive the culture scaffold.

5. Device according to any one of the preceding claims, the culture scaffold (20) being removably received in the culture chamber.

6. Device according to any one of the preceding claims, comprising a body (15) defining a housing (50) in which is arranged the culture scaffold (20), and an insert (51) to close at least partially said housing, this insert being preferably screwed into the body (15).

7. Device according to claims 3 and 6, the insert (51) defining said collector (56).

8. Device according to claims 6 and 7, the insert (51) comprising an end lip (65) engaged on the seal (70), and preferably extending onto a part only of the height of the seal.

9. Device according to claim 8, the seal (70) having a radially outer surface inclined with respect to its axis of symmetry, notably a rectangular trapezoidal shaped section, such that the axial tightening of the insert induces, by wedge effect, a radial tightening of the seal.

10. Device according to any one of claims 6 to 9, the insert (51) having a central cut-out (52) to receive an end fitting (59) for connecting a departure tubing, this end fitting preferably being screwed into the insert.

11. Device according to any one of the preceding claims, the network (80) of micro-channels extending along a plane perpendicular to the axis (X) of the culture scaffold (20).

12. Device according to one of the preceding claims, comprising a block having a housing (50) defining at least partially the culture chamber, having a bearing face (17) against which a bottom plate (16) is directly mounted, the network (80) of micro-channels being formed between said face and the bottom plate, the micro-channels preferably being formed as hollows on said bearing face (17).

13. Device according to any one of the preceding claims, the culture scaffold (20) comprising a plurality of parallel channels (400), extending between the inlet and outlet faces of the culture scaffold, these channels preferably having a circular section, the channels preferably being parallel to the axis (X) of the culture scaffold.

14. Device according to any one of the preceding claims, the outlets of the network (80) of micro-channels having axial symmetry or second order rotational symmetry at least, better fourth order at least, even better eighth order at least.

15. Device according to any one of the preceding claims, the network (80) of micro-channels comprising at least two stages (82, 84, 85) of multiple ramifications.
